(19) United States
(12) Patent Application Publication
(10) Pub. No.: US 2022/0082565 A1
(43) Pub. Date: Mar. 17, 2022

(54) THE FGF19-CHOLESTYRAMINE (F-CME) TEST AS A TWO-STAGE METHOD FOR ROUTINE CANCER SCREENING

(71) Applicant: UNIVERSITY OF CENTRAL FLORIDA RESEARCH FOUNDATION, INC., ORLANDO, FL (US)

(72) Inventors: Michael ROHR, Orlando, FL (US); Trina RUDESKI-ROHR, Orlando, FL (US); Deborah A. ALTOMARE, Orlando, FL (US)

(21) Appl. No.: 17/410,658

(22) Filed: Aug. 24, 2021

Related U.S. Application Data
(60) Provisional application No. 63/069,321, filed on Aug. 24, 2020.

(51) Int. Cl. G01N 33/574

(52) U.S. Cl. CPC G01N 33/57492; G01N 2333/50

(57) ABSTRACT

Disclosed is a two-staged, generalized cancer screening concept modeled closely after a suppression test. The test is blood based, and screens for elevated levels of Fibroblast Growth Factor 19 (FGF19), a cancer biomarker expressed across a majority of cancers, via standard enzyme-linked immunosorbent assay (ELISA). Unlike other biomarker-based blood tests that rely on a single-blood draw, the F-CME test validates cancer with a follow-up test using oral cholestyramine as the “suppression” drug.

### BACKGROUND

Cancer represents one of the greatest burdens on global healthcare. In America, cancer is the second leading cause of death and more than 600,000 Americans are projected to die of cancer in 2020. Despite recent advances in treatment and management, the overall mortality rate remains unacceptably high, and this is due to the nature of cancer itself. Specifically, most cancers are out of sight and remain asymptomatic until tumor growth impacts the normal bodily functions or have metastasized. In cases where early symptoms are present, they are usually non-specific and conflated with other non-serious conditions which subsequently delays diagnosis. Ultimately, many cancers present at late pathologic stages which results in a worse prognosis regardless of management strategy. Thus, the best way to further reduce cancer mortality would be to implement a routine screening test that is generally acceptable, minimally invasive, widely available, and reliable enough to detect a wide array of cancer types before symptom onset.

Blood tests have been looked at to fill this clinical need as they are standard part of the healthcare system, minimally invasive, and can be collected in a variety of settings and performed multiple times if needed. In practice, blood-based cancer screening tests have proven somewhat unreliable and even controversial due to the drawbacks and non-specific nature of the signature being detected, often confounding positive readings. This mainly comes down to the fact that cancers do not “truly” produce any unique markers because they are derived from normal tissues, as well as the inability of a single blood draw to account for the heterogenous nature of both cancer and the patient population being tested.

The approach can mostly be solved by the addition of a follow-up test that can be used to validate whether a blood marker is originating from a tumor and not something else. This test would ideally exploit the difference in the production of a certain marker between normal and tumor tissue using an existing pharmacological agent. By utilizing a more robust protein marker and corresponding pharmacological “suppressor,” the two-step suppression test concept can be modified and applied in a general cancer screening context, resulting in superior sensitivity and specificity relative to a single blood test.

### BRIEF DESCRIPTION OF THE DRAWINGS

The present invention is illustrated by way of example, and not by way of limitation, in the figures of the accompanying drawings.

The following figures are illustrative only and are not intended to be limiting.

FIG. 1 A is a graph showing that FGF19 expression is highly tissue restricted and only shows detectable levels in the testis, small intestines, and gall bladder in normal tissue

FIG. 1 B is a graph showing that median FGF19 expression is significantly elevated in 23 of the 27 cancers (˜83%) assayed by The Cancer Genome Atlas (TCGA), including all of the most common cancer types

FIG. 1 C is a graph showing elevated expression in cancer can be due to copy number amplification.

FIG. 1 D is the list of cancers and their abbreviations shown in FIG. 1 A-C

FIG. 2 are graphs showing that FGF19 expression in many cancers is both specific and sensitive. The graphs show that pancreatic, colorectal, esophageal, ovarian, and stomach cancer will be better candidates for this test.

FIG. 3 A is a graph showing that FGF19 expression has good overall clinical utility in cancer.

FIG. 3 B are graphs showing FGF19 expression related to cancer pathologic stage. The graphs show that FGF19 expression is elevated in later stages.

FIG. 3 C is an overall survival graph showing FGF19 expression related to cancer prognosis.

FIG. 4 is a diagram showing FGF19 role as a regulator of enterohepatic recirculation of bile and inhibitor of de novo hepatic bile acid synthesis from cholesterol.

FIG. 5 is a diagram showing the signaling pathway leading to the secretion of FGF 19. Bile acids are actively pumped into the intestinal cell via the apical sodium-bile acid transporter (ASBT) and activate the steroid Farnesoid-X-Receptor (FXR) transcription factor, which stimulates the production and secretion of FGF19.

FIG. 6 A-B are figure showing that FGF19 expression and serum levels fall dramatically upon acute treatment of humans with a bile acid sequestrant (cholestyramine, colestipol, or colesevelam).

FIG. 7 A-B is a diagram showing the concept of F-CME second stage testing. A negative result will show a decrease of FGF19 levels after cholestyramine treat, and a positive result will show no change in FGF19 levels after cholestyramine.

FIG. 8 is a flow chart showing the stages of F-CME testing. The chart shows the options for low, normal, or elevated FGF19 in patients after stage I and stage II.

FIG. 9 is a compilation of several studies of baseline serum FGF19 levels. The levels do vary but seem to fall within 60-400 pg/mL.

FIG. 10A-D are graphs of the bioinformatic analysis revealing FGF19 as a candidate biomarker in CRC.

FIG. 11A-B are graphs showing that FGF19 is a predictive biomarker in CRC.

FIG. 12A-D are graphs and a western blot showing ectopic FGF19 expression and secretion is linked in vitro.

FIG. 13 A-D are graphs showing FGF19 is secreted in blood and reflects tumor burden in vivo.

FIG. 14 A-E are graphs showing that FGF19 expression is uncoupled from FXR in CRC.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “about” means plus or minus 1%, 2%, 3%, 4%, 5%, 6%, 7%, 8%, 9%, or 10% of the number to which reference is being made.

“Biomarker” as used herein means a genetic variant, of any size or type, contained within an individual's genome that is associated with a disease or condition, such as drug response, or any molecule derived therefrom, including a DNA molecule expressing the genetic variant, an RNA molecule transcribing the genetic variant, or a polypeptide that the genetic variant encodes.

“Biological sample”, as used herein, includes blood or blood component (e.g. plasma, serum, red blood cells, peripheral immune cells) or other fluid sample (tears, urine, semen, vaginal fluid, wound exudate, sweat, sputem, etc.). Blood is typically obtained by venipuncture.

“Cholestyramine” as used herein is synonymous to colestyramine or trade names Questran, Questran Light, Cholybar, Olestyr. It is a large, highly positively charged anion exchange resin that binds to negatively charged anions such as bile acids.

“Cancer screening” as used herein in means looking for cancer before symptoms appear. Screening includes, but is not limited to, physical exams and history, laboratory tests, imaging procedures, and genetic tests.

“Cancer progression” as used herein in means any increase of growth or invasiveness of tumor cells. It also describes phenotypical changes that increase aggressiveness and malignant potential.

“Cancer therapy” as used herein refers to a therapy for treating a detected cancer including radiotherapy, chemotherapy, surgery (e.g. tissue resection), immunotherapy, kinase inhibition, monoclonal antibody therapy (e.g., bevacizumab or cetuximab) or a combination thereof. A given cancer therapy to be administered can be determined by a person of ordinary skill in the art depending upon the status of cancer in a subject, for example, the subject to be treated may be evaluated, in particular, for the state of the subject's cancer, subjects overall health, age and desired aggressiveness of the therapy.

“Elevated levels” as used herein in means higher amounts of the nucleic acids or polypeptides of the biomarker that indicates or predicts a need for medical intervention or disease.

“Enzyme-linked immunosorbent assay (ELISA)” as used herein means a solid phase-based assay technique designed for detecting and quantifying analytes such as peptides, proteins, antibodies, and hormones. Other names, such as enzyme immunoassay (EIA), are also used to describe the same technology. In a typical example, an antigen (target macromolecule) is immobilized on a solid surface (e.g., microplate) and then complexed with an antibody that is linked to a reporter enzyme. Detection is accomplished by measuring the activity of the reporter enzyme via incubation with the appropriate substrate to produce a measurable product. The most crucial element of an ELISA is a highly specific antibody-antigen interaction.

“Fibroblast Growth Factor 19 (FGF19)” as used herein in means a protein that in humans is encoded by the FGF19 gene. It functions as a hormone, regulating bile acid synthesis, with effects on glucose and lipid metabolism. The amino acid sequence is given by NP_005108.

“Hepatocellular carcinoma” as used herein in means the most common type of primary liver cancer in adults and mostly occurs in people with cirrhosis of the liver.

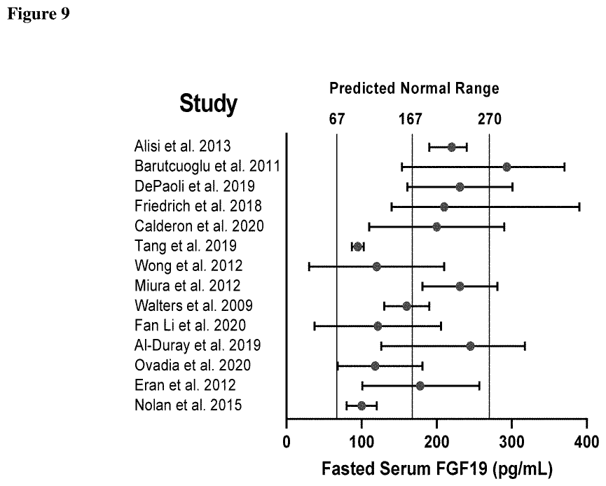

“Normal range” as used herein refers to an amount of FGF19 in a biological sample from a normal subject. Baseline serum FGF19 levels do vary but seem to fall within 60-400 pg/mL (FIG. 9).

“Normal subject” as used herein refers to a subject that lacks a disease or disorder in question. For example, a normal subject does not have cancer.

“Post-curative” as used herein in means the monitoring stage after a patient is cleared of having the primary and secondary tumors. The monitoring can last anywhere from 5 to 10 years and comprises of screening for recurrence of the cancer.

“Serum” as used herein in means blood plasma from which the clotting proteins have been removed or any clear bodily fluid.

“Treat”, “treating” or “treatment of” as used herein refers to providing any type of medical management to a subject. Treating includes, but is not limited to, administering a composition to a subject using any known method for purposes such as curing, reversing, alleviating, reducing the severity of, inhibiting the progression of, or reducing the likelihood of a disease, disorder, or condition or one or more symptoms or manifestations of a disease, disorder or condition.

### Overview

Low compliance with screening recommendations is a major contributor to Colorectal Cancer (CRC) morbidity and mortality. In the US, about one third of eligible Americans do not follow these recommendations, a statistic projected to rise following the recent inclusion of the 45-50 age demographic. Cost, preparation, invasiveness, and sample handling represent key barriers to screening. In a survey of patients refusing colonoscopy, almost all participants cited a desire for more convenient alternatives, especially a blood-based test. Because serum biomarkers may originate from other sources besides malignancy, reliance on a single reading can make such results suspect. Rather, a practical solution could be a multi-stage test that can distinguish these readings as either physiologic or malignant. For example, described here is a test that exploits the nature of a potential cancer biomarker being uncoupled from its normal physiologic regulatory mechanism, which, when pharmacologically suppressed, will have little impact on levels if malignancy is present. Presented in this disclosure is a two-step suppression-based test would be effective as a blood-based CRC detection method.

Through comprehensive informatics and literature meta-analyses, Fibroblast Growth Factor 19 (FGF19) was identified as a candidate for this test. FGF19 has properties such as tissue-restrictive and context dependent production, short circulatory half-life, reduced serum levels in comorbid diseases, minimal hepatic clearance, and potential for urine-based detection in addition to blood. In CRC, FGF19 expression demonstrates greater predictive strength than other cancers, is associated with clinicopathology, and circulating levels could indicate tumor responsiveness to relevant clinical inhibitors like Fisogatinib. Important for the two-step suppression test, FGF19 production in the distal intestines depends on enterohepatic recirculation of bile acids (BAs) which can be blocked by BA sequestrants such as cholestryamine (CM), a commonly prescribed cholesterol lowering drug. Previous reports and data show FGF19 production in CRC is BA-independent, supporting a suppression-based test using CM as an innovative solution to the pitfalls of current modalities.

### Description of Embodiments

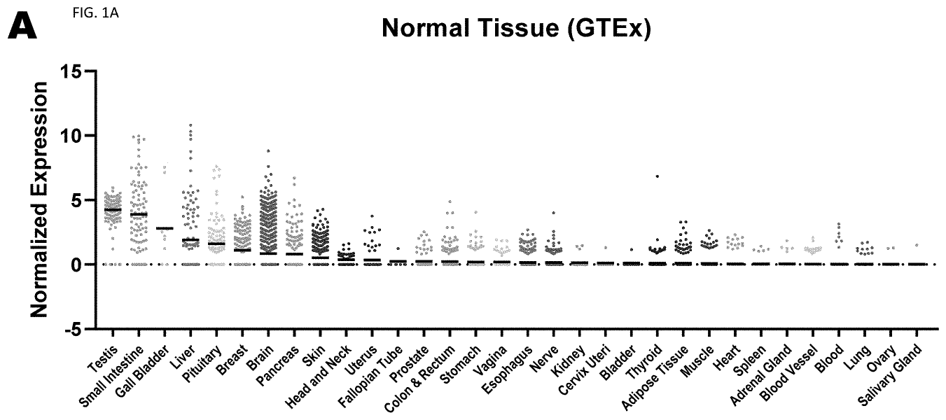

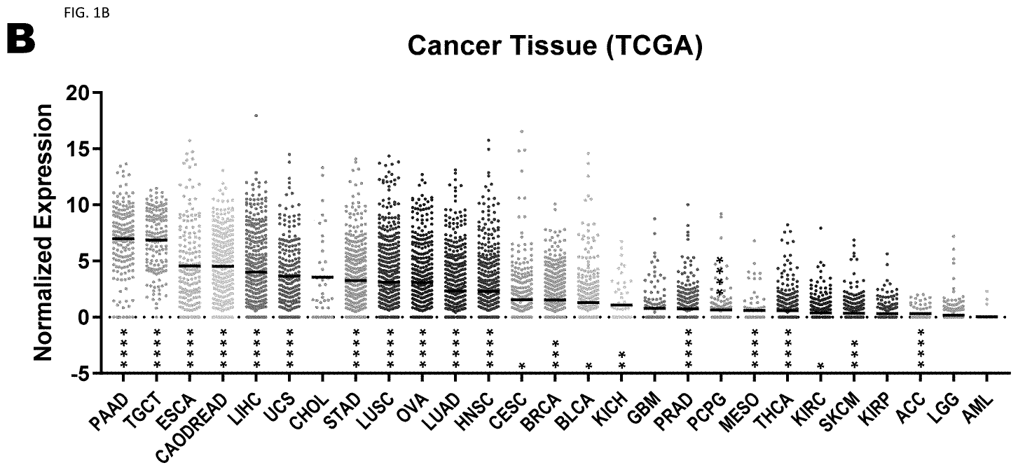

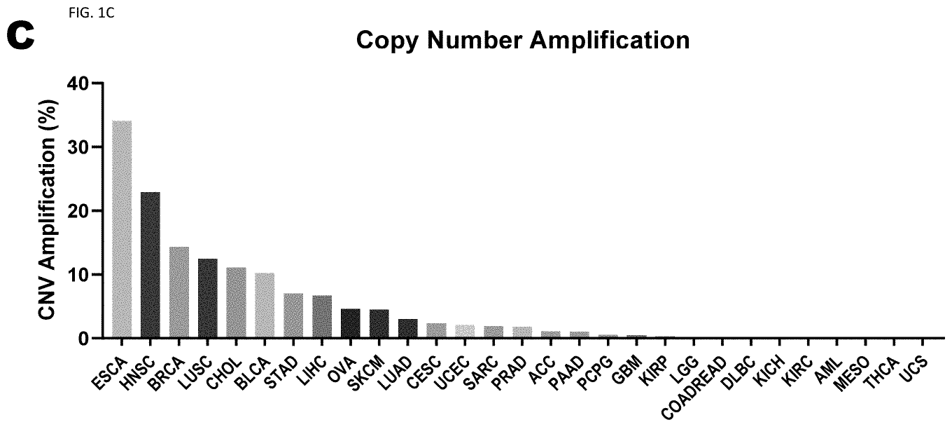

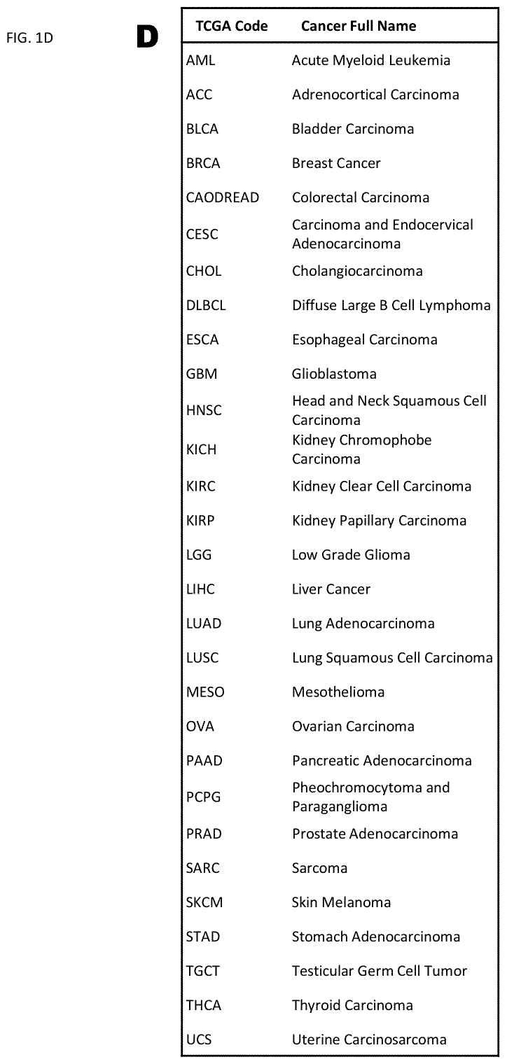

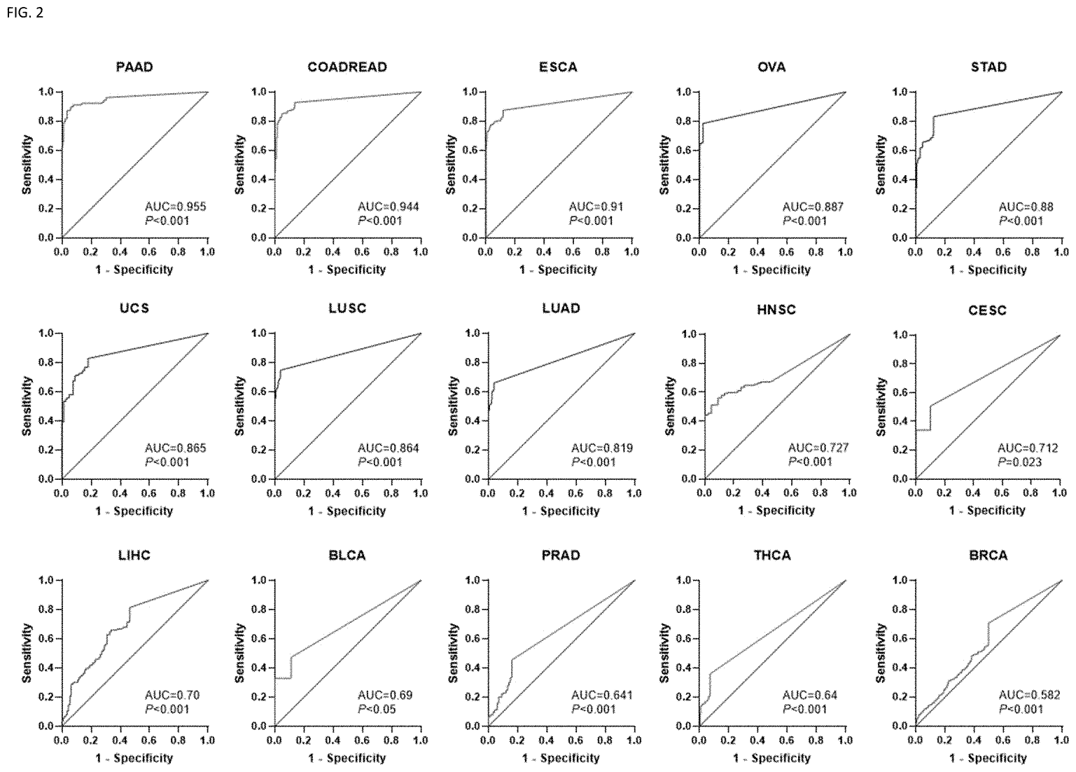

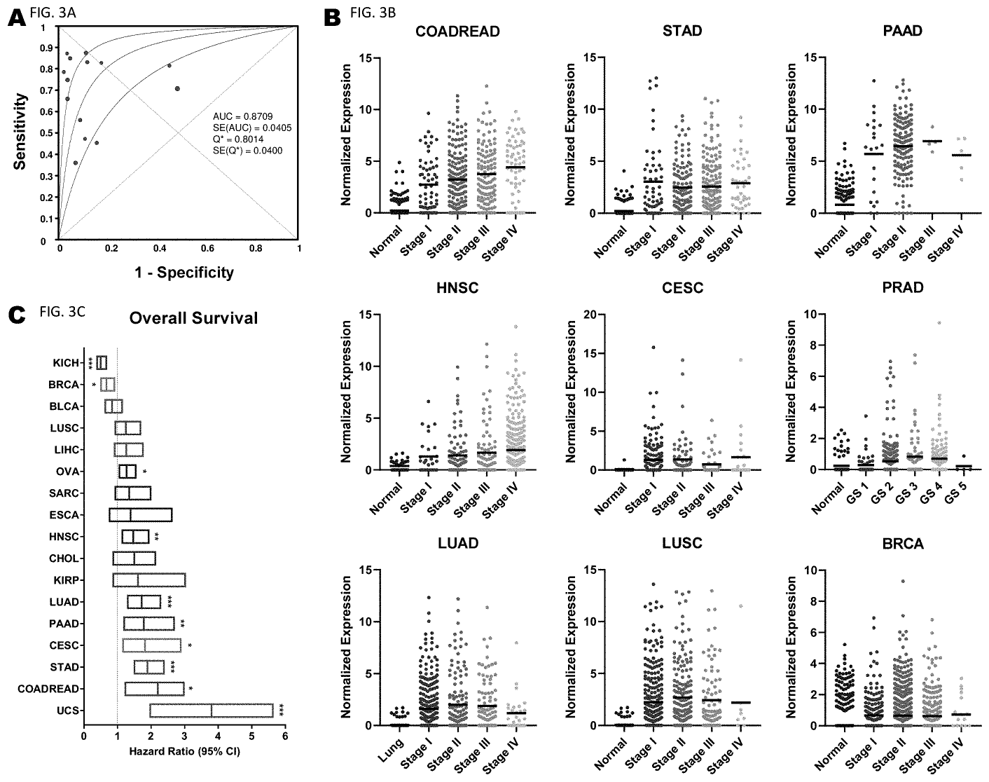

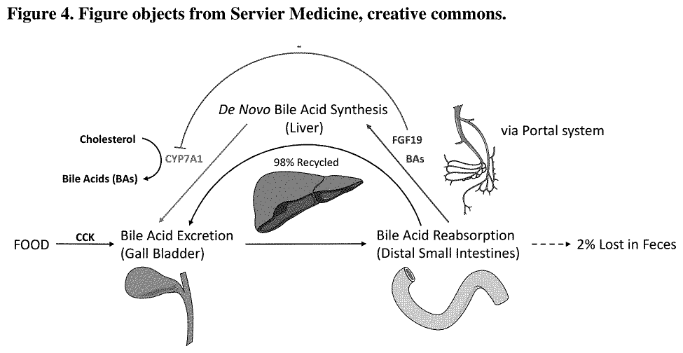

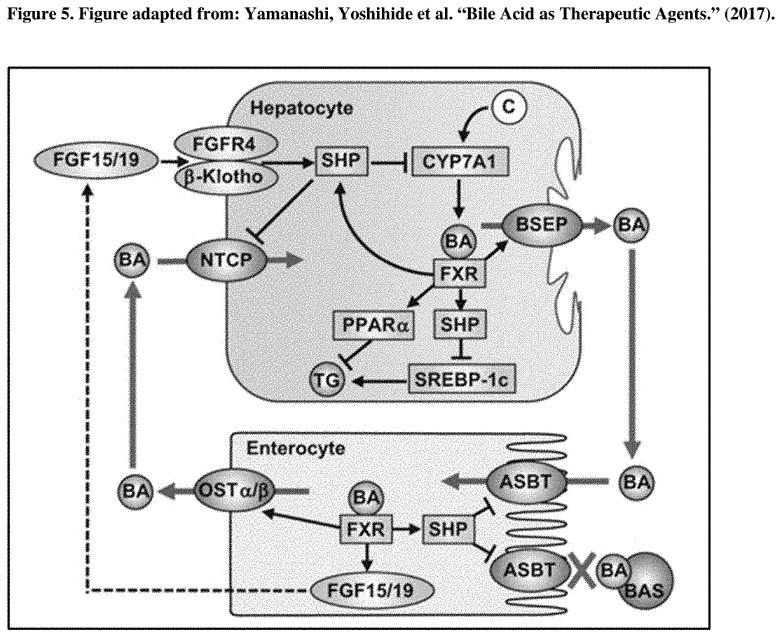

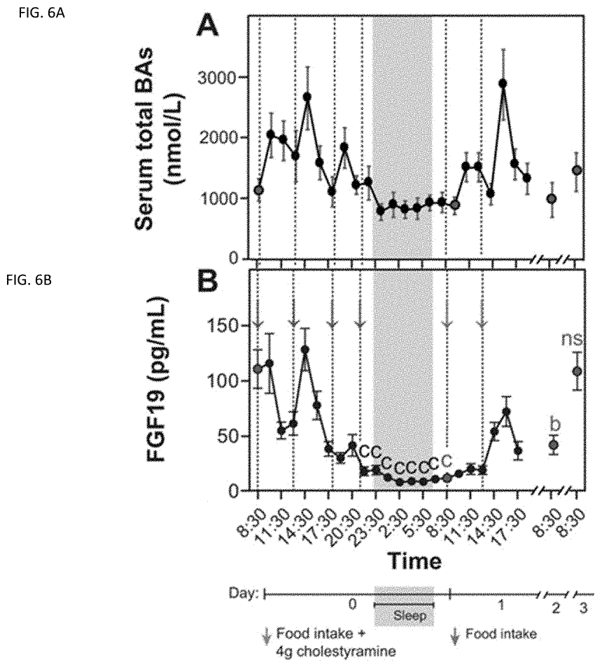

The present disclosure is based on the discovery that median Fibroblast Growth Factor 19 (FGF19) expression is significantly elevated in 23 of the 27 cancers (˜83%) assayed by The Cancer Genome Atlas (TCGA), including all the most common cancer types (FIG. 1B). Moreover, depending on the cancer evaluated, elevated expression can either be due to copy number amplification (FIG. 1C, FGF19 falls within the commonly amplified 11q13.3 amplicon) or just regular transcriptional overexpression. Importantly, FGF19 expression in many cancers is both specific and sensitive (FIG. 2 and Table 1), shows good overall clinical utility in cancer (FIG. 3A), and is related to cancer pathologic stage (FIG. 3B) and prognosis (FIG. 3C).

TABLE 1 shows that FGF19 expression in many cancers is both specific and sensitive.

TABLE 1

| Study | Expression Cutoff | Specificity | Sensitivity | TP | FP | FN | TN | Cancer (n) | Normal (n) |
|---|---|---|---|---|---|---|---|---|---|
| PAAD | 3.927 | 0.964 | 0.872 | 156 | 6 | 23 | 161 | 179 | 167 |
| COADREAD | 1.405 | 0.951 | 0.849 | 338 | 15 | 60 | 293 | 398 | 308 |
| ESCA | 0.3112 | 0.882 | 0.874 | 571 | 21 | 82 | 161 | 653 | 182 |
| OVA | 0.2778 | 0.977 | 0.785 | 336 | 2 | 92 | 86 | 428 | 88 |
| STAD | 0.2473 | 0.879 | 0.831 | 344 | 21 | 70 | 153 | 414 | 174 |
| UCS | 0.308 | 0.821 | 0.828 | 197 | 14 | 41 | 64 | 238 | 78 |
| → LUSC | 0.311 | 0.962 | 0.747 | 424 | 11 | 143 | 277 | 567 | 288 |
| → LUAD | 0.2088 | 0.962 | 0.660 | 385 | 11 | 199 | 277 | 584 | 288 |
| → HNSC | 0.965 | 0.909 | 0.560 | 291 | 4 | 229 | 40 | 520 | 44 |
| CESC | 0.2712 | 0.900 | 0.507 | 155 | 1 | 151 | 9 | 306 | 10 |
| → LIHC | 0.284 | 0.536 | 0.814 | 302 | 51 | 69 | 59 | 371 | 110 |
| BLCA | 0.2821 | 0.889 | 0.472 | 192 | 1 | 215 | 8 | 407 | 9 |
| → PRAD | 0.256 | 0.840 | 0.454 | 225 | 16 | 271 | 84 | 496 | 100 |
| → THCA | 0.2051 | 0.928 | 0.359 | 184 | 20 | 328 | 259 | 512 | 279 |
| → BRCA | 0.2442 | 0.503 | 0.707 | 777 | 89 | 322 | 90 | 1099 | 179 |
| Total | — | — | — | — | — | — | — | 7172 | 2304 |

→ Indicates what cancers have literature to support that expression translates into serum levels

FGF19 itself has been deemed an essential oncogene in multiple cancer settings and is known to actively promote tumorigenesis through interacting with cognate receptor, Fibroblast Growth Factor Receptor 4 (FGFR4, [2-11]). This is important because a few newly synthesized therapeutics targeting FGF19 (FGF401, [17]) and FGFR4 (fisogatinib, [18]) are currently undergoing clinical trials for treating certain cancers, namely Hepatocellular carcinoma (HCC). In CRC, FGF19 expression demonstrates greater predictive strength than other cancers, is associated with clinicopathology, and circulating levels could indicate tumor responsiveness to relevant clinical inhibitors like Fisogatinib. Pre-clinical models have also demonstrated the efficacy of targeted FGF19 and/or FGFR4 therapeutics (15,19,20). Therefore, elevated serum FGF19 levels that are eventually traced back to a tumor may provide valuable insight into possible chemosensitivity/chemoresistance, enabling a precision medicine approach for improved curative treatment.

In one embodiment, disclosed is a FGF19-Cholestyramine (F-CME) test that involves a two-staged, generalized cancer screening concept. The test is blood based, and screens for elevated levels of Fibroblast Growth Factor 19 (FGF19), a cancer biomarker expressed across a majority of cancers. In a preferred embodiment, a standard enzyme-linked immunosorbent assay (ELISA) is used for measuring FGF19 levels. Unlike other biomarker-based blood tests that rely on a single-blood draw, the F-CME test validates cancer with a follow-up test using oral cholestyramine as the “suppression” drug.

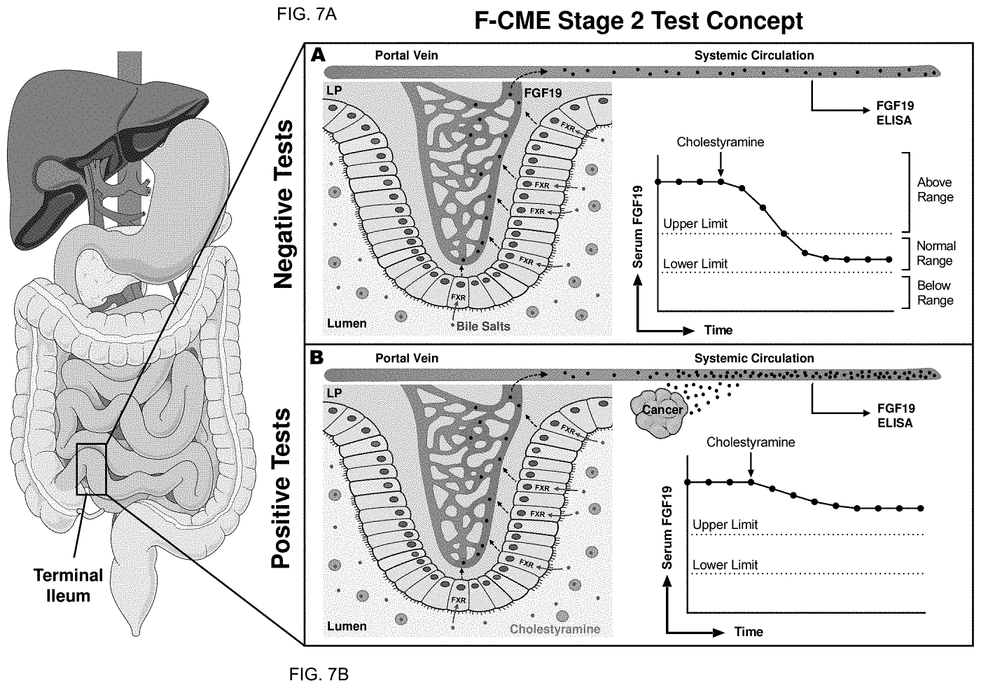

Collectively, the theory behind the F-CME test is as follows: A majority of cancers express and secrete FGF19, resulting in elevated baseline FGF19 in fasting patients. In order to validate that elevated serum FGF19 is indeed cancerous, cholestyramine will be administered in order to “suppress” serum FGF19 levels that are normal in origin. Cancers secrete FGF19 independent of bile acids and thus are unaffected by cholestyramine whereas FGF19 coming from the terminal ileum will be suppressed. Thus, serum FGF19 that remains elevated despite cholestyramine treatment means that the source is coming from a site other than the terminal ileum which is highly suggestive of cancer (FIG. 7).

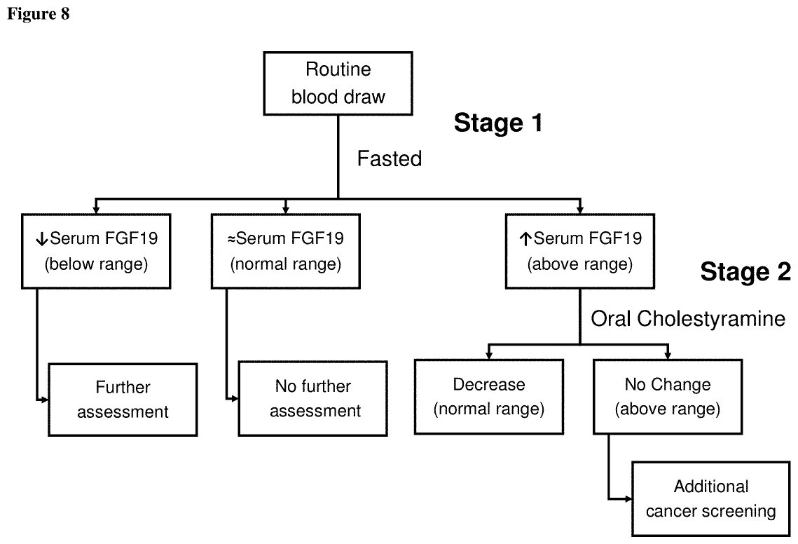

Stage 1: A routine blood test screening for serum FGF19 would show either low, normal, or high levels. A low and normal reading would be expected for a healthy individual, although a low reading could indicate the presence of a secondary disease (this will be up to the physician's discretion whether to further pursue). For patients with an elevated reading, they are recommended the secondary test, or stage 2 (FIG. 8).

Stage 2: Following oral cholestyramine treatment, serum FGF19 is lowered to normal or below normal is expected in a healthy individual and could indicate that the patient was not fasted during stage 1 or point to a natural variation. In these cases, no further action is needed. If serum FGF19 remains elevated, then cancer is suspected, and the physician may recommend further follow-up screening to determine the origin of the readings (FIG. 8).

In certain embodiments, the F-CME test is employed in a routine manner as part of normal blood draw. The F-CME test could also be indicated in high-risk patients (e.g. family history, genetic mutations, behavioral, etc.) or in cancer patients for pre- and post-curative treatment monitoring.

The F-CME test is administered in two stages. Stage 1 is the routine blood draw where fasted serum FGF19 is determined directly using a standard ELISA. If results from stage 1 are positive, patients are enrolled in stage 2. During stage 2, a patient is rescheduled for a follow-up blood draw as early as possible and given a packet consisting of 3-4 mini-packs containing 4 grams of cholestyramine powder each (16). Patients are instructed to mix the entire contents of each mini-pack with breakfast, lunch, and dinner (for three) and liquid before bed (for four packs) the day before the rescheduled blood draw. The following day, the patient should remain fasted until blood is re-drawn and sent for analysis. This should occur ideally between 7 AM and 2 PM to minimize natural diurnal variations in FGF19 levels. The embodiments described in this disclosure presents an innovative solution to the pitfalls of conventional blood tests for Colorectal cancer (CRC) and contextualizes the application of this concept for other cancers that lack screening alternatives.

### FGF19

Fibroblast growth factor 19 is a protein that in humans is encoded by the FGF19 gene. It functions as a hormone, regulating bile acid synthesis, with effects on glucose and lipid metabolism. Reduced synthesis, and blood levels, may be a factor in chronic bile acid diarrhea and in certain metabolic disorders.

The protein encoded by this gene is a member of the fibroblast growth factor (FGF) family. FGF family members possess broad mitogenic and cell survival activities and are involved in a variety of biological processes including embryonic development cell growth, morphogenesis, tissue repair, tumor growth and invasion. This growth factor is a high affinity, heparin dependent ligand for FGFR4. The orthologous protein in mouse is FGF15, which shares about 50% amino acid identity and has similar functions. Together they are often referred to as FGF15/19.

FGF19 has important roles as a hormone produced in the ileum in response to bile acid absorption. Bile acids bind to the farnesoid X receptor (FXR), stimulating FGF19 transcription. Several FXR/bile acid response elements have been identified in the FGF19 gene. Human FGF19 transcripts have been shown to be stimulated approximately 300-fold by physiological concentrations of bile acids including chenodeoxycholic acid, glycochenodeoxycholic acid and obeticholic acid in explants of ileal mucosa.

FGF19 is frequently amplified in human cancers. Amplification of the FGF19 genomic locus was found in liver cancer, breast cancer, lung cancer, prostate cancer, bladder cancer, and esophageal cancer, among others. Targeting FGF19 inhibits tumor growth in colon cancer cells and hepatocellar carcinoma. Increase in FGF19 correlates with tumor progression and poorer prognosis of hepatocellular carcinoma.

Baseline serum FGF19 levels do vary but seem to fall within 60-400 pg/mL (FIG. 9). Abnormal serum FGF19 levels are greater than about 400 pg/mL, and after treatment with cholestyramine, serum FGF19 levels should return to 60-400 pg/mL in a healthy patient.

### Administrations

Routes of administration of a therapeutic compound or composition of the disclosure include but are not limited to parenteral (including subcutaneous, intraperitoneal, intrasternal, intravenous, intraarticular injection, infusion, intradermal, and intramuscular); or oral; pulmonary, mucosal (including buccal, sublingual, vaginal, and rectal); topical, transdermal, and the like. Oral administration is a preferred route of administration.

The amounts of cholestyramine used in therapeutic methods of the disclosure will vary according to various factors including but not limited to the specific compounds being utilized, the particular compositions formulated, the mode of application, the site of administration, the age and the body weight of the subject and the condition of the subject to be treated, and ultimately will be decided by the attending physician. Conventional dosing determination tests can be used to ascertain the optimal administration rates for a given protocol of administration. Doses utilized in prior clinical applications for cholestyramine will provide guidelines for preferred dosing amounts for the methods of the present disclosure.

### Assays

Assays according to the disclosure are biochemical tests that detect, in a specific and qualitative, semi-quantitative or quantitative manner, the presence of a target analyte in a sample. Specific binding assays of this type rely on the ability of a specific-binding “capture” molecule to bind to the target analyte to be detected or measured without appreciable binding to any other component of a complex sample containing numerous other macromolecules. Commonly, these types of tests are referred to as ligand binding tests, or, for example, immunoassays. A detection method is used to determine the presence and extent of the binding which occurs, therefore the assay involves a label or other means to produce a detectable or measurable signal in response to this binding. Many different labels or other mechanisms are available to permit detection of the signal through different means, such as detection of radiation, color change or intensity, fluorescence, chemiluminescence, enzyme activity, physical agglutination or clumping, and the like. Steps in a typical assay of this type usually involve (1) sample collection and preparation; (2) analyte capture; and (3) detection. Examples of assays that may be implemented to detect analytes useful in accord with the assay and method embodiments herein are further described below.

Sample collection can be performed according to any of the methods known in the art for collecting a bodily fluid, cellular, tissue or other sample. Any sample which contains or is suspected of containing the target analyte to be detected in the assay can be used.

Fluid (liquid, semi-liquid, gelatinous, and the like) samples commonly are be collected by aspiration using a needle or collection in a vessel or by swab. Fluid samples optionally are treated prior to assay by, for example, mixing, filtration, dilution or serial dilution, or centrifugation (e.g., to remove cells, cellular debris, or other particulates) to produce a better or cleaner sample for assay.

Capture of the analyte can be performed in solution or on a substrate using any convenient capture molecule. Most commonly, antibodies, such as polyclonal or monoclonal antibodies, or binding fragments thereof are used as the capture molecule, however any convenient capture molecule is suitable for use with the invention as long as it binds to the analyte specifically and with high affinity and specificity. Preferably, the capture molecule is able to bind the analyte at nanomolar concentrations or less, more preferably at picomolar or attomolar concentrations. Antibody substitute capture molecules such as aptamers, aptides, affibodies, affimers, avimers, and the like can serve as capture molecules, as well as receptors, specific binding partners, ligands, and the like.

Assays according to embodiments of the disclosure can be configured to operate in any convenient format known in the art. For example, the assay can be competitive or non-competitive, or a sandwich assay, and can be performed in solution (liquid phase) or on any of several known substrates. Some immunoassays can be carried out simply by mixing the reagents and sample and making a physical measurement, including newer “mix-and-measure” assays, which do not require the separation of bound from free ligand, for example bead-based assays. Such assays are called homogenous assays or less frequently non-separation assays. Multi-step assays are often called separation assays or heterogeneous assays. Commonly used assay types include radioimmune assays (RIA), immunoradiometric assays (IRA), enzyme-linked immunosorbant assays (ELISA), agglutination assays, precipitation or sedimentation assays, lateral flow (immuno)assays (LFIA), or blotting assays such as dot blots, western blots, and the like, each using any of the known capture molecules and detection systems. The assays according to embodiments of the disclosure can be automated using high throughput automatic analyzer instruments or robotic methods.

Many assays are named for the detection system which they employ, for example radioimmunoassays use a radioactive label, magnetic immunoassays use a magnet for separation, fluorescent immunoassays use a fluorescent label, while ELISA tests use an enzyme-substrate reaction to develop a detectable color. Fluorescent resonance energy transfer (FRET) systems and proximity ligation assays are other examples of assays that are described based on the detection system. Any of these assay types are contemplated for use with embodiments of the invention. Further description of detection methods is found below. Liquid phase ligand binding assays that rely on specifically binding capture molecules also include nucleic acid hybridization assays, which typically use an intercalating fluorescent dye that emits fluorescence via secondary structure conversion, molecular beacon capture of specific nucleic acid sequences, or real-time RT-qPCR using a molecular beacon or fluorophore intercalating dye.

A very simple form of assay is the “mix-and-measure” type or homogenous assay, in which the reagents are mixed together and the signal is read. Specific examples of such assays are described in, for example, Kreisig et al., Scientific Reports 4:5613, 2014; Miskolci et al., Meth. Mol. Biol. 1172:173-184, 2014; Wang et al., Biosensors and Bioelectronics 26(2):743-747, 2010; Luu et al., http://www.kiko-tech.co.jp/products/intellicyt/ique_screener/intellicyt_hybridoma.pdf; Edelhoch, H., Hayaishi, O., and Teply, L.: The Preparation and Properties of a Soluble Disphosphopyridine Nucleotide Cytochrome C Reductase, J Biol Chem 197, 97, 1952; Mahler, H., Sarkar, N., Vernon, L., and Alberty, R.: Studies on Diphosphopyridine Nucleotide-Cytochrome c Reductase II. Purification and Properties, J Biol Chem 199, 585, 1952; Stowell et al., Anal. Biochem. 15:58-64, 2016; Einhorn et al., EPMA J. 6:23. 2015. Other homogenous assays that may be implemented with the system and method embodiments described herein include:
1. Fluorescence Polarization Immunoassay (FPIA) Maragas, Toxins, 2009 1:196-207;
2. Enzyme Multiplied Immunoassay (EMIT), Zherdev et al., Analytica Chimica Acta, 1997 347:131-138;
3. Dynamic Light Scattering. Nanoparticles conjugated with a capture molecule will bind to analyte contained in the sample creating a particle-biomolecular complex. These complexes can be detected using dynamic light scattering. See U.S. Pat. Nos. 8,883,094 and 9,005,994 and Liu et al. J. Am. Chem. Soc. 2008, 130, 2780-2782; for examples of detecting analytes using dynamic light scattering and metal particles;
4. Homogenous Temperature and Substrate Resolved Chemiluminescence Multi-analyte Immunoassay, See Kang et al., Analyst, 2009, 134:2246-2252; and
5. AlphaLISA assay (Perkin-Elmer, Waltham, Mass.). Ullman, E. F. et al. Luminescent oxygen channeling assay (LOCI): sensitive, broadly applicable homogeneous immunoassay method. Clin. Chem. 42, 1518-1526 (1996). McGiven, J. A. et al. A new homogeneous assay for high throughput serological diagnosis of brucellosis in ruminants. J. Immunol. Methods. 337, 7-15 (2008). This assay uses two different beads (alpha donor bead and AlphaLISA acceptor bead) that when both are bound to analyte, the acceptor bead can emit light at a certain wavelength upon excitation.

Detection of the binding of capture molecule to target analyte can be achieved by any of a large number of known methods. Any of these methods are contemplated for use with embodiments of the invention. Examples of labeling and detection methods include, but are not limited to, radioactive isotope, enzyme-substrate, colorimetric and visual, fluorescence, chemiluminescence, magnetic, molecular beacons, and the like.

Traditional competitive (homogenous) assays involve a competition reaction in which the target analyte in the sample competes for binding to a specific binding capture molecule (such as an antibody or aptamer, for example) with a labeled analyte reagent. After binding, the amount of the labeled, unbound analyte is measured. The more analyte present in the sample, the less labelled analyte reagent is able to bind to the capture molecule, therefore the amount of labeled, unbound analyte is inversely proportional to the amount of analyte in the sample. In a competitive (heterogenous) assay, unlabeled target analyte from the sample competes for binding to the capture molecule with a labeled analyte reagent as described above, however the labeled unbound analyte reagent is separated or washed away and the remaining labeled bound analyte is measured. Any of these types of assays, or variations thereof as known in the art, are contemplated for use with embodiments of the disclosure.

Commonly, the capture molecule is immobilized on a membrane, a reaction vessel surface or on suspended beads such as agarose beads, and detection is achieved using a labeled secondary binding molecule, such as an antibody or aptamer, that specifically binds to the primary capture molecule or to another binding region on the target analyte. If the capture molecule is immobilized on beads, separation and detection can be achieved using flow cytometry, magnetic separation, and the like. In addition, binding of the capture molecule and target analyte can be detected in solution without immobilization on a substrate.

In a typical non-competitive assay, the target analyte binds to a specific capture molecule that is labeled. After separating the unbound labeled capture reagent, the bound material is measured. The intensity of the signal is directly proportional to the amount of unknown analyte in the original sample. Alternatively, the assay is performed in a “sandwich” format where the target analyte binds to the capture molecule (which usually is bound to a surface for ease of separation) and labeled secondary capture molecule also binds to the target analyte. The amount of labeled capture molecule on the surface is then measured. The label intensity is directly proportional to the concentration of the analyte because labelled antibody will not bind forming a “sandwich” if the analyte is not present in the unknown sample.

Sandwich format binding ligand affinity assays can be performed with different detection methods. Typically, these assays are performed as solid-phase assays, where the target analyte is “sandwiched” between an immobilized capture molecule and a labeled capture molecule, each capture molecule binding to a different, non-overlapping epitope or binding area of the analyte. Immobilization allows the user to remove unbound substances from the bound analyte prior to detection with the labeled capture molecule. The primary capture molecule can be immobilized on any surface, for example the surface of the testing vessel (e.g., a multiwell plate), beads, a dipstick, filters, or column resins. The capture molecules (primary and secondary (labelled)) can be selected individually from antibodies, antibody substitutes, receptors, aptamers, nucleic acids, or any specific binding molecule. Most commonly these assays use an enzyme detection system, but any detection system can be used. Further labels and detection systems are discussed below. An exemplary sandwich-type assay can be performed using a biotinylated aptamer or antibody capture molecule, immobilized on a streptavidin plate or beads. Sample containing the target analyte is incubated in a buffered solution with the immobilized capture molecule and then is washed away, leaving bound target analyte. A secondary capture molecule, such as an antibody or antibody substitute, then is incubated in a buffered solution with the bound target. The sandwich complexes are detected directly, by detecting the label on the secondary capture molecule, or indirectly using a labeled antibody that binds to the secondary capture molecule. These assays are known in the art and can be modified as necessary by a person of skill, including determining optimum concentrations of the reagents, and the like.

Competitive assays can be designed on a number of platforms and using various detection methods, however a two-step assay is preferable when greater sensitivity is required or the available sample size is small. In a typical two-step assay, sample containing the target analyte is exposed to immobilized capture molecules that bind the analyte. The immobilized analyte, bound to the capture molecules, then is exposed to a solution containing conjugated (labeled) analyte at a high concentration. This conjugated analyte saturates any of the immobilized capture molecules which are not bound to target analyte from the sample. Before equilibrium is reached and the previously bound target analyte can be displaced, the conjugate solution is removed. The amount of label bound to the immobilized capture molecules is inversely proportional to the amount of analyte present in the sample.

“Pull-down assay” refers to an assay which comprises removal of a target from solution. This removal occurs when a capture molecule in solution or suspension is mixed with the sample containing the target analyte and specifically binds to it. The capture molecule is labeled or bound to a substrate which allows the bound material to precipitate, agglutinate or otherwise be physically separated, for example using simple gravity, a magnet, centrifugation, and the like. In an agglutination assay, capture molecules that are bi- or multimeric- (i.e., that possess two or more specific binding areas, like an antibody) or substrates bearing multiple capture molecules, bind to the target analyte, forming large complexes that clump, precipitate, or agglutinate in the solution and fall to the bottom of the testing vessel. These large complexes can be seen with the naked eye if large enough and contain a visible color, for example, or can be seen with the aid of a microscope. In some embodiments, the clumps also contain a label that can be detected by other means, or the clumped material can be analyzed by chromatographic means. Latex agglutination involves latex particles, preferably colored particles, which are coated with bound capture molecules, which form complexes in the presence of the target analyte. Pull-down assays are convenient methods to determine whether a physical interaction between the target analyte and the capture molecule has taken place, i.e., to determine the presence of the analyte or as a semi-quantitative assay to determine relative amounts of the analyte.

Lateral flow tests also known as lateral flow immunochromatographic assays, are simple devices intended to detect the presence (or absence) of a target analyte in sample (matrix) without the need for specialized and costly equipment. Typically, these tests are used for medical diagnostics either for home testing, point of care testing, or laboratory use. These tests are based on a series of capillary beds through and across which the sample fluid migrates from a sample area or sample pad, across defined areas that contain various reagents. A typical assay uses a conjugate pad, in which the conjugated capture molecule which binds specifically to the target analyte is located. Upon binding, the captured analyte continues to flow laterally to a second area where a secondary capture molecule binds and immobilizes the conjugate-analyte complex in a relatively small area. Once the complexes accumulate, the conjugate's label, usually a colored particle, becomes more concentrated and hence detectable, often by the accumulation of color. Lateral flow tests of this type can operate as either competitive or sandwich assays.

General background information regarding lateral flow immunoassay systems is provided in Lateral Flow Immunoassay, Raphael C. Wong and Harley Y. Tse (Editors), 2009, Humana Press, a part of Springer Science+Business Media, LLC. (Library of Congress Control Number 2008939893) and U.S. Pat. No. 8,011,228.

Methods for Treating Disease and Disorders Associated with Elevated FGF19

The disclosure further provides a method of treating CRC, hepatocellular carcinoma, other cancers, or other diseases and disorders using any drugs, compounds, small molecules, proteins, antibodies, nucleotides, and pharmaceutical compositions thereof, that are capable of normalizing levels of FGF19.

Effective dosages and administration regimens can be readily determined by good medical practice and the clinical condition of the individual subject. The frequency of administration will depend on the pharmacokinetic parameters of the active ingredient(s) and the route of administration. The optimal pharmaceutical formulation can be determined depending upon the route of administration and desired dosage. Such formulations may influence the physical state, stability, rate of in vivo release, and rate of in vivo clearance of the administered compounds.

Depending on the route of administration, a suitable dose may be calculated according to body weight, body surface area, or organ size. Optimization of the appropriate dosage can readily be made by those skilled in the art in light of pharmacokinetic data observed in human clinical trials. The final dosage regimen will be determined by the attending physician, considering various factors which modify the action of drugs, e.g., the drug's specific activity, the severity of the damage and the responsiveness of the patient, the age, condition, body weight, sex and diet of the patient, the severity of any present infection, time of administration and other clinical factors.

The embodiments in this disclosure are further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

It is to be noted that throughout this application various publications and patents are cited. The disclosures of these publications are hereby incorporated by reference in their entireties into this application in order to describe fully the state of the art to which this invention pertains.

### Examples

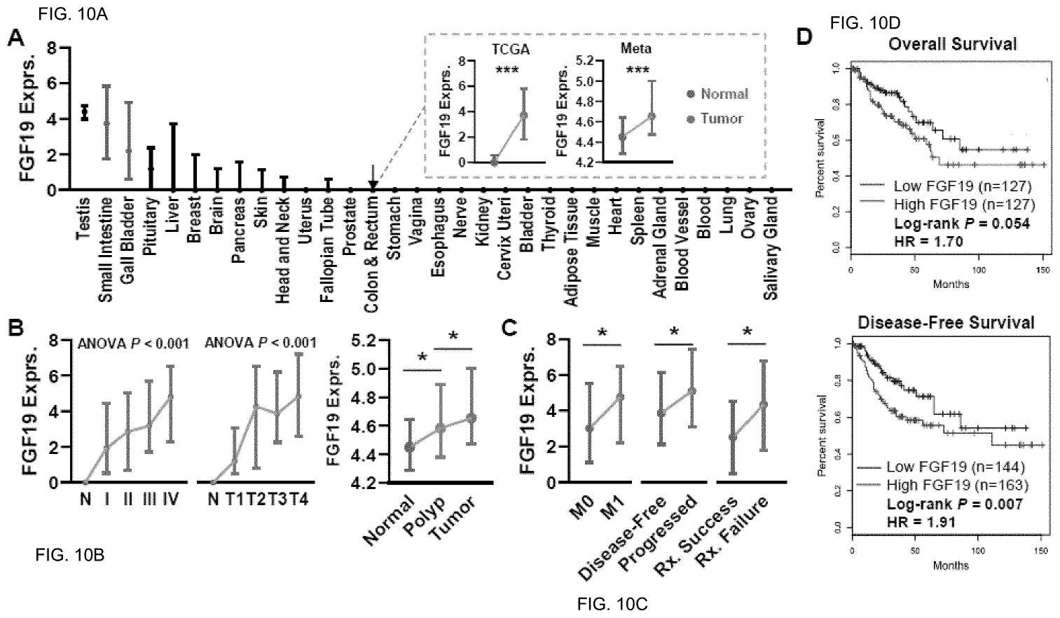

Example 1. Bioinformatics shows FGF19 is a candidate CRC biomarker. FGF19 was determined to be a putative biomarker for this proposal based on transcriptomic profiles in normal colon and CRC tissue. First, FGF19 expression is tissue restrictive based on expression profiles from Genotype-Tissue Expression (GTEx) dataset (FIG. 10A)[38]. Expression was present only in the small intestines and gall bladder (blue) with none detected in colon (arrow) as expected. In contrast, FGF19 overexpression was readily detected in CRC tissue within both TCGA-COADREAD (RNAseq)[39] and a 12-study Affymetrix Metadataset (Microarray) we constructed (E-MTAB-6043) (FIG. 10A, insets). FGF19 expression also increased stepwise with pathologic stages (FIG. 10B, left), was elevated in polyps (FIG. 10B, right), and was associated with prognostic indicators (FIG. 10C) and reduced survival metrics (FIG. 10D). If FGF19 expression was proportional to amounts secreted by tumors into blood, then these results indicate that circulating levels could reflect tumor progression and unfavorable clinicopathology.

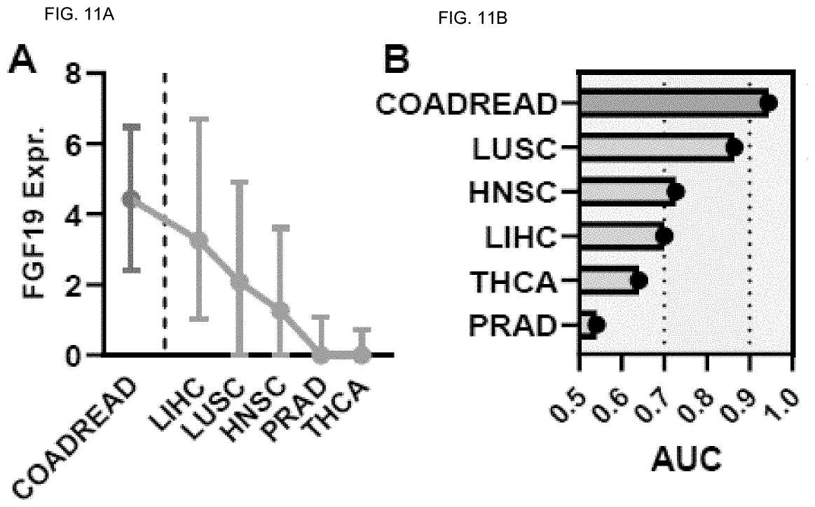

Example 2. FGF19 expression is a strong predictor of CRC. FGF19 overexpression has been studied in various tumor types,[3,4,6,8,9] especially hepatocellular carcinoma (LIHC)[5,7] However, compared to cancers with supporting literature, the degree of FGF19 overexpression in CRC is more substantial (FIG. 11A) based on TCGA data [39-44] Likewise, FGF19 was also found to be a more predictive marker for CRC relative to other cancers based on receiver operator characteristic (ROC) analysis, demonstrating an area under the curve (AUC) of 0.95 with an 85% sensitivity and 94% specificity maxima (FIG. 11B).

Example 3. Serum levels of FGF19 are reduced in common conditions. A common pitfall of conventional markers is that abnormal serum levels may not indicate cancer and instead could be confounded by other underlying conditions[45,46]. An ideal serum marker would be reduced in these cases as to strengthen suspicion of an aberrant elevated reading. A literature review indicated serum levels of FGF19 are significantly reduced in many common and comorbid diseases, such as Crohn's Disease (Table 2), but elevated only in a few contexts.

TABLE 2 shows FGF19 is elevated in cancer states and reduced in other common or comorbid disease states

TABLE 2
Diseases and Drugs

| Reduced Serum FGF19 | Elevated Serum FGF19 |
|---|---|
| Sepsis (22) | Hepatocellular Carcinoma (5, 7) |
| IBS-D (23) | All Thyroid Cancer Types (4, 6) |
| Type 2 Diabetes (24-26) | Non-Small Cell Lung Carcinoma (9) |
| Obesity/BMI (27, 28) | Prostate Cancer (3) |
| Non-Alcoholic Fatty Liver | Head and Neck Squamous Cell |
| Disease (29) | Carcinoma (8) |
| Active Crohn's Disease (30) | Ursodeoxycholic acid -FXR |
| | agonist (10) |
| Bile Acid Malabsorption | Extrahepatic Cholestasis (2) |
| Diarrhea (31) | |
| Non-Alcoholic Steatohepatitis (32) | Chronic hemodialysis (11) |

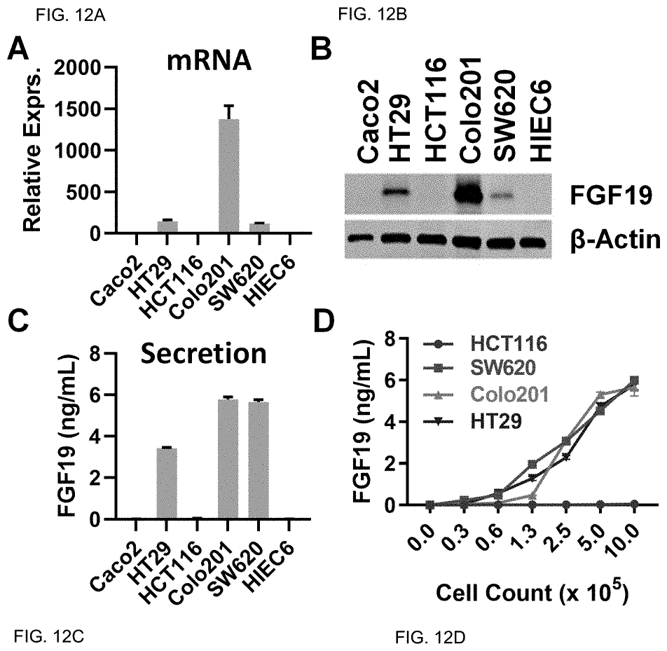

Example 4. FGF19 expression in Colorectal Cancer (CRC) is linked with secretion in vitro. As an endocrine factor, we hypothesized that FGF19-expressing tumor cells would secrete FGF19 into their surroundings. To test this hypothesis and contextualize our in silico findings, we investigated whether FGF19 secretion was dependent on mRNA and protein expression in a panel of CRC cell lines. Out of 5 cell lines, 3 (HT29, SW620, and Colo201) were strong, 1 (HCT116) was weak, and 1 (Caco2) was negative for FGF19 (FIG. 12A) expression. Similar results were observed at the protein level (FIG. 12B). No expression was detected in the normal intestinal cell line HIEC6. Importantly, media levels of FGF19 reflects expression patterns and even occur in serum-free media (FIG. 12C), suggesting that malignant FGF19 production is constitutive and likely bile acid (BA)-independent. Furthermore, we found that secretory levels increased with cell density (FIG. 12D).

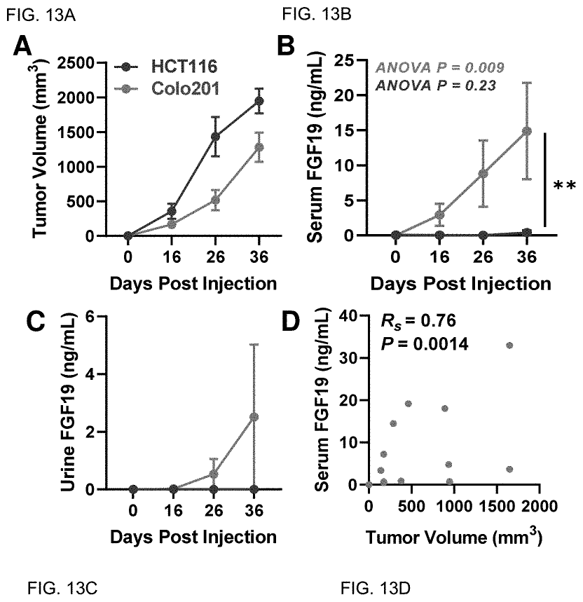

Example 5. CRC cell-derived xenografts (CDXs) secrete FGF19 into mouse blood. Based on our in vitro data, we fully expected that FGF19 secreted by CRC tumors would become circulatory and be detected in blood. As no reports have explored this, we piloted a longitudinal in vivo study using a subcutaneous (s.c.) CDX model of CRC. Briefly, 8 male and female NOD SCID Gamma (NSG) mice were randomly assigned to two groups (n=4) either receiving s.c. injections of HCT116 (FGF19-weak) or Colo201 (FGF19-positive) cells. Following a 2-week incubation period, blood was collected every week for 3 weeks and assayed for serum FGF19. Urine collected opportunistically was also assayed to test if FGF19 is renally filtered. Despite tumors establishing for both groups (FIG. 13A), FGF19 was only detected in the serum of mice harboring Colo201, but not HCT116, CDXs as expected (FIG. 13B). This was also true for urine (FIG. 13C), but enough was only collected from two mice per group so more samples are required for verification. Strikingly, serum FGF19 concentrations were directly associated with tumor volume (FIG. 13D).

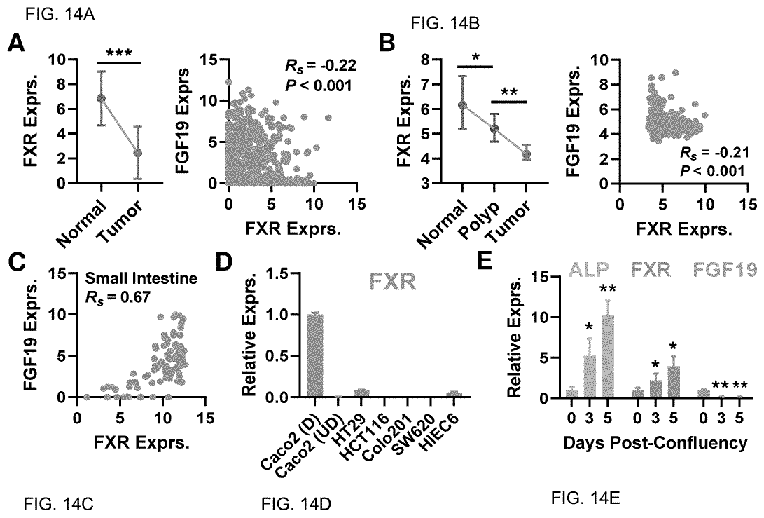

Example 6. FGF19 expression in CRC is likely Bile Acid (BA)-independent. The CMS test is predicated on malignant FGF19 production being BA-independent. We postulated that the observed ectopic and constitutive expression patterns of FGF19 indicated uncoupling from physiologic regulation by FXR. Indeed, the first indication of this stems from known expression patterns of both components. For example, FXR is known to be downregulated early in CRC and is a potent tumor suppressor. Conversely, FGF19 is a known CRC oncogene, and we show is overexpressed in tumors. Unsurprisingly, FXR downregulation was a feature of CRC and was negatively associated with FGF19 expression patterns in both TCGA (FIG. 14A) and Meta-dataset (FIG. 14B) cohorts. This is in contrast to the positive association found in normal small intestine samples within the GTEx dataset (FIG. 14C). Like FGF19, FXR expression patterns were recapitulated in cell lines, showing almost complete absence in all but HT29, normal-like differentiated (D) Caco2, and normal HIEC6 cells (FIG. 14D). Importantly, FGF19 was expressed in FXR-negative cell lines and was regulated opposite to FXR upon differentiation of HT29 cells (FIG. 14E), indicating uncoupling.

### REFERENCES

1. Galman, C., Angelin, B. & Rudling, M. Pronounced variation in bile acid synthesis in humans is related to gender, hypertriglyceridaemia and circulating levels of fibroblast growth factor 19: Rapid Communication: Bile acid synthesis and FGF19. J. Intern. Med. 270, 580-588 (2011).
2. Schaap, F. G., van der Gaag, N. A., Gouma, D. J. & Jansen, P. L. M. High expression of the bile salt-homeostatic hormone fibroblast growth factor 19 in the liver of patients with extrahepatic cholestasis. Hepatol. Baltim. Md 49, 1228-1235 (2009).
3. Nagamatsu, H. et al. FGF19 promotes progression of prostate cancer. The Prostate 75, 1092-1101 (2015).
4. Zhang, X. et al. Increased Expression of FGF19 Contributes to Tumor Progression and Cell Motility of Human Thyroid Cancer. Otolaryngol. Neck Surg. 154, 52-58 (2016).
5. Vijayvargiya, P. et al. Performance Characteristics of Serum C4 and FGF19 Measurements to Exclude the Diagnosis of Bile Acid Diarrhoea in IBS-Diarrhoea and Functional Diarrhoea.

Aliment. Pharmacol. Ther. 46, 581-588 (2017).
6. Motylewska, E. et al. Alteration in the serum concentrations of FGF19, FGFR4 and βKlotho in patients with thyroid cancer. Cytokine 105, 32-36 (2018).
7. Maeda, T. et al. Serum fibroblast growth factor 19 serves as a potential novel biomarker for hepatocellular carcinoma. BMC Cancer 19, 1088 (2019).
8. Gao, L. et al. FGF19 amplification reveals an oncogenic dependency upon autocrine FGF19/FGFR4 signaling in head and neck squamous cell carcinoma. Oncogene 38, 2394-2404 (2019).
9. Li, F. et al. Enhanced autocrine FGF19/FGFR4 signaling drives the progression of lung squamous cell carcinoma, which responds to mTOR inhibitor AZD2104. Oncogene 39, 3507-3521 (2020).
10. Ovadia, C. et al. Ursodeoxycholic acid enriches intestinal bile salt hydrolase expressing Bacteroidetes in cholestatic pregnancy. Sci. Rep. 10, 3895 (2020).
11. Marchelek-Myśliwiec, M. et al. Chronic Kidney Disease Is Associated with Increased Plasma Levels of Fibroblast Growth Factors 19 and 21. Kidney Blood Press. Res. 44, 1207-1218 (2019).
12. Inagaki, T. et al. Fibroblast growth factor 15 functions as an enterohepatic signal to regulate bile acid homeostasis. Cell Metab. 2, 217-225 (2005).
13. Zhang, J. H. et al. Potent stimulation of fibroblast growth factor 19 expression in the human ileum by bile acids. Am. J. Physiol. Gastrointest. Liver Physiol. 304, G940-948 (2013).
14. Yamanashi, Y., Takada, T. & Suzuki, H. Bile Acid as Therapeutic Agents. in Bile Acids in Gastroenterology (eds. Tazuma, S. & Takikawa, H.) 61-90 (Springer Japan, 2017). doi:10.1007/978-4-431-56062-3_5.
15. Desnoyers, L. R. et al. Targeting FGF19 inhibits tumor growth in colon cancer xenograft and FGF19 transgenic hepatocellular carcinoma models. Oncogene 27, 85-97 (2008).
16. Sjöberg, B. G., Straniero, S., Angelin, B. & Rudling, M. Cholestyramine treatment of healthy humans rapidly induces transient hypertriglyceridemia when treatment is initiated. Am. J. Physiol.-Endocrinol. Metab. 313, E167-E174 (2017).
17. Weiss, A. et al. FGF401, A First-In-Class Highly Selective and Potent FGFR4 Inhibitor for the Treatment of FGF19-Driven Hepatocellular Cancer. Mol. Cancer Ther. 18, 2194-2206 (2019).
18. Subbiah, V. & Pal, S. K. Precision Oncology for Hepatocellular Cancer: Slivering the Liver by FGF19-FGFR4-KLB Pathway Inhibition. Cancer Discov. 9, 1646-1649 (2019).
19. Turkington, R. C. et al. Fibroblast growth factor receptor 4 (FGFR4): a targetable regulator of drug resistance in colorectal cancer. Cell Death Dis. 5, e1046 (2014).
20. Lang, L. & Teng, Y. Fibroblast Growth Factor Receptor 4 Targeting in Cancer: New Insights into Mechanisms and Therapeutic Strategies. Cells 8, (2019).
21. Degirolamo, C., Sabba, C. & Moschetta, A. Therapeutic potential of the endocrine fibroblast growth factors FGF19, FGF21 and FGF23. Nat. Rev. Drug Discov. 15, 51-69 (2016).
22. Tang, X. et al. Serum fibroblast growth factor 19 as a predictor for early stage of gastrointestinal-liver dysfunction in pediatric patients with sepsis. https://www.researchsquare.com/article/rs-2417/v1 (2019) doi:10.21203/rs.2.11560/v1.
23. Wong, B. S. et al. Increased bile acid biosynthesis is associated with irritable bowel syndrome with diarrhea. Clin. Gastroenterol. Hepatol. Off. Clin. Pract. J. Am. Gastroenterol.

Assoc. 10, 1009-1015.e3 (2012).
24. Barutcuoglu, B. et al. Fibroblast growth factor-19 levels in type 2 diabetic patients with metabolic syndrome. Ann. Clin. Lab. Sci. 41, 390-396 (2011).
25. DePaoli, A. M. et al. FGF19 Analog as a Surgical Factor Mimetic That Contributes to Metabolic Effects Beyond Glucose Homeostasis. Diabetes 68, 1315-1328 (2019).
26. Tang, M.-J. et al. Serum fibroblast growth factor 19 and endogenous islet beta cell function in type 2 diabetic patients. Diabetol. Metab. Syndr. 11, 79 (2019).
27. Gómez-Ambrosi, J. et al. FGF19 and FGF21 serum concentrations in human obesity and type 2 diabetes behave differently after diet- or surgically induced weight loss. Clin. Nutr. 36, 861-868 (2017).
28. Calderon, G. et al. Ileo-colonic delivery of conjugated bile acids improves glucose homeostasis via colonic GLP-1-producing enteroendocrine cells in human obesity and diabetes. EBioMedicine 55, 102759 (2020).
29. Eren, F. et al. Preliminary evidence of a reduced serum level of fibroblast growth factor 19 in patients with biopsy-proven nonalcoholic fatty liver disease. Clin. Biochem. 45, 655-658 (2012).
30. Nolan, J. D. et al. Diarrhea in Crohn's Disease: Investigating the Role of the Ileal Hormone Fibroblast Growth Factor 19. J. Crohns Colitis 9, 125-131 (2015).
31. Walters, J. R. F. et al. A new mechanism for bile acid diarrhea: defective feedback inhibition of bile acid biosynthesis. Clin. Gastroenterol. Hepatol. Off. Clin. Pract. J. Am. Gastroenterol.

Assoc. 7, 1189-1194 (2009).
32. Alisi, A. et al. Association between Serum Atypical Fibroblast Growth Factors 21 and 19 and Pediatric Nonalcoholic Fatty Liver Disease. PLoS ONE 8, e67160 (2013).
33. Han, S. et al. Circadian control of bile acid synthesis by a KLF15-Fgf15 axis. Nat Commun 6, 7231 (2015).
34. Bradley, E. L. & Galambos, J. T. Diagnosis of gastrinoma by the secretin suppression test. Surg Gynecol Obstet 143, 784-788 (1976).
35. Koelfat, K. V. K. et al. The portal-drained viscera release fibroblast growth factor 19 in humans. Physiol Rep 4, (2016).
36. Johansson, H. et al. Circulating Fibroblast Growth Factor 19 in Portal and Systemic Blood. J Clin Exp Hepatol 8, 162-168 (2018).
37. Reiche, M. et al. Fibroblast Growth Factor 19 Serum Levels: Relation to Renal Function and Metabolic Parameters. Horm Metab Res 42, 178-181 (2010).
38. Carithers, L. J. et al. A Novel Approach to High-Quality Postmortem Tissue Procurement: The GTEx Project. Biopreservation and Biobanking 13, 311-319 (2015).
39. Cancer Genome Atlas Network. Comprehensive molecular characterization of human colon and rectal cancer. Nature 487, 330-337 (2012).
40. Ally, A. et al. Comprehensive and Integrative Genomic Characterization of Hepatocellular Carcinoma. Cell 169, 1327-1341.e23 (2017).
41. Cancer Genome Atlas Research Network. Comprehensive genomic characterization of squamous cell lung cancers. Nature 489, 519-525 (2012).
42. Cancer Genome Atlas Network. Comprehensive genomic characterization of head and neck squamous cell carcinomas. Nature 517, 576-582 (2015).
43. Abeshouse, A. et al. The Molecular Taxonomy of Primary Prostate Cancer. Cell 163, 1011-1025 (2015).
44. Cancer Genome Atlas Research Network. Integrated genomic characterization of papillary thyroid carcinoma. Cell 159, 676-690 (2014).
45. Rule, A. H., Goleski-Reilly, C., Sachar, D. B., Vandevoorde, J. & Janowitz, H. D. Circulating carcinoembryonic antigen (CEA): Relationship to clinical status of patients with inflammatory bowel disease. Gut 14, 880-884 (1973).
46. Fukuda, I., Yamakado, M. & Kiyose, H. Influence of smoking on serum carcinoembryonic antigen levels in subjects who underwent multiphasic health testing and services. Journal of Medical Systems 22, 89-93 (1998).

What is claimed is:

1. A method comprising
(i) detecting an amount of FGF19 in a first biological sample from a subject;
(ii) treating the subject with cholestyramine if the amount of FGF19 in the biological sample is above a normal range;
(iii) detecting an amount of FGF19 in a second biological sample obtained from the subject after step (ii); and
(iv) identifying the subject as positive for cancer if the amount of FGF19 in the second biological sample is not at a normal range.

2. The method of treating of claim 1, wherein the cholestyramine treatment comprises of 1 day.

3. The method of claim 2, wherein the cholestyramine dose comprises about 3-4 packs containing about 4 grams of cholestyramine powder for oral administration.

4. The method of claim 3, wherein the cholestyramine is administered with a meal 72 hours or less before step (iii).

5. The method of claim 1, further comprising administering a cancer therapy if the subject is identified as positive for cancer.

6. The method of claim 5, wherein the method can be used to monitor cancer progression during treatment.

7. The method of claim 6, wherein steps (ii)-(iv) are repeated.

8. The method of claim 6, wherein the method can be used for post-curative cancer monitoring.

9. The method of claim 1, wherein the first and/or second biological sample is urine, blood, or a blood component.

10. The method of claim 9, wherein the blood component is serum.

11. The method of claim 1, wherein the subject has fasted for at least four hours before step (i).

12. The method of screening of claim 1, wherein detecting the FGF19 levels comprises immunostaining, immunoprecipitation, immunoelectrophoresis, Immunoblotting, BCA assay to quantify protein concentrations, Western blot, Spectrophotometry, and enzyme-linked immunosorbent assays (ELISA).

14. The method of claim 1, wherein the cancer comprises liver, pancreatic, colorectal, esophageal, ovarian, or stomach cancer.

15. The method of claim 14, wherein the liver cancer comprises hepatocellular carcinoma.
